(19) United States
(12) Patent Application Publication
(10) Pub. No.: US 2021/0220545 A1
(43) Pub. Date: Jul. 22, 2021

(54) NON-CONTACTING, HIGH ACCURACY PRESSURE SENSING FOR MEDICAL CASSETTE ASSEMBLIES

(71) Applicant: Johnson & Johnson Surgical Vision, Inc., Santa Ana, CA (US)

(72) Inventors: Eric N. Anderfaas, Westminster, CA (US); Matthew Flowers, Aliso Viejo, CA (US); Nicolas Welche, Irvine, CA (US); Derek Bissell, Westminster, CA (US); Travis Cochran, Costa Mesa, CA (US); David Spargur, Fullerton, CA (US)

(21) Appl. No.: 17/124,369

(22) Filed: Dec. 16, 2020

Related U.S. Application Data
(60) Provisional application No. 62/949,416, filed on Dec. 17, 2019.

(51) Int. Cl. A61M 1/00

(52) U.S. Cl. CPC A61M 1/774; A61M 2205/3331; A61M 2205/02; A61M 2205/3327

(57) ABSTRACT

A system for pressure measurement within a surgical system is disclosed. The system comprises a pressure sensitive disc in communication with at least one applied pressure a magnetic field generator for generating at least one first magnetic field, and at least one sensor for measuring at least one second magnetic field, wherein the at least one first magnetic field at least partially creates the at least second magnetic field; and wherein the at least one sensor produces signal indicative of the distance between the at least one sensor and the at least one second magnetic field.

### CROSS-REFERENCE TO RELATED APPLICATIONS

This application claims the benefit under 35 U.S.C. § 119(e) of U.S. Provisional patent Application No. 62/949,416, filed Dec. 17, 2019, which is incorporated herein by reference in its entirety.

### BACKGROUND OF THE INVENTION

### Field of Technology

The present invention relates generally to the sensing of pressure within a surgical cassette and, more specifically, to the sensing of fluidic pressure in a surgical cassette using a non-contacting, high accuracy pressure sensing apparatus and method.

### Description of the Background

The use of surgical consoles often requires pressure sensing of fluidics on single use medical packs (also called cassettes). In typical use environments, single use cassettes may have drops of liquid, glove marks or other debris on the exterior of the cassette which may complicate the operation of traditional pressure sensing techniques, especially in the communication between a surgical console and a surgical cassette. Indeed, surgical operations today using a console often demand high accuracy pressure sensing of fluids even when contamination is present on the exterior of the single use cassette.

More specifically, traditional single use cassettes use inexpensive pressure sensors built into the cassettes, making it difficult to obtain the necessary pressure sensing accuracy, especially with a low-cost sensor. Moreover, such pressure sensors often require an electrical connection between the single use cassette and the interfacing surgical console. Other single use cassettes use a low-cost pressure diaphragm on the cassette with a console mounted Linear Variable Differential Transformer (LVDT) to measure the deflection of the pressure diaphragm with either a low rate spring pushing the LVDT against the surface of the pressure diaphragm or a magnet coupling the LVDT to the surface of the diaphragm, or a combination of both a spring and magnet. The spring force and/or friction force associated with movement of the LVDT sensing element reduces the accuracy and repeatability of this type system. Additional known systems have used laser triangulation displacement sensors to measure the deflection of a pressure diaphragm. However, these laser type systems often have technical issues with liquid or debris on the pressure sensing diaphragm surface which can lead to spurious pressure readings.

Thus, there exists a need for the sensing of fluidic pressure in a surgical cassette using a non-contacting, high accuracy pressure sensing apparatus and method.

### SUMMARY

A pressure measurement system for use with a surgical system is disclosed, comprising a pressure sensitive disc in communication with at least one applied pressure, a magnetic field generator for generating at least one first magnetic field, and at least one sensor for measuring at least one second magnetic field, wherein the at least one first magnetic field at least partially creates the at least second magnetic field, and wherein the at least one sensor produces signal indicative of the distance between the at least one sensor and the at least one second magnetic field.

The present invention discloses a single use cassette pressure sensing system using a cassette mounted pressure sensing diaphragm and a console mounted eddy current displacement sensor. The eddy current displacement sensor may measure the deflection of the pressure sensing diaphragm which may yield a noncontact pressure sensing system with high accuracy pressure measurement which is immune to liquid, glove marks or debris on the surface of the diaphragm. The present invention provides for very high accuracy of pressure measurements, a non-fluid contact pressure sensing, and no external force generating elements such as a spring or friction contacting the pressure diaphragm.

A method for determining a pressure is disclosed, comprising, providing a first magnetic field to a metallic body to induce an at least one second magnetic field, sensing the at least one second magnetic field using at least one sensor, and determining the distance between the metallic body and the at last one sensor based on the at least one second magnetic field. A displacement sensor may be mounted to the console, not the single use cassette, and may not require an electrical connection between the cassette and the console.

### DESCRIPTION OF THE DRAWINGS

The accompanying drawings are included to provide a further understanding of the invention and are incorporated in and constitute a part of this specification. The drawings illustrate disclosed embodiments and/or aspects and, together with the description, serve to explain the principles of the invention, the scope of which is determined by the claims.

FIG. 1A is a schematic illustrating an eye treatment system in which a cassette is coupled to an eye treatment probe with an eye treatment console under one embodiment;

FIG. 1B is a schematic illustrating a surgical eye treatment console under another exemplary embodiment;

FIG. 2 is a functional block diagram of an exemplary cassette system for an eye treatment system under one embodiment;

FIG. 3 is a schematic illustrating a cassette under another exemplary embodiment;

FIG. 4A is an illustration of an exemplary diaphragm under one embodiment;

FIG. 4B is an illustration of experimental data on an exemplary diaphragm under one embodiment;

FIG. 5A is an illustration of an exemplary diaphragm under one embodiment;

FIG. 5B is an illustration of experimental data on an exemplary diaphragm under one embodiment;

FIG. 5C is a cross-sectional illustration of an exemplary diaphragm under one embodiment;

FIG. 5D is an illustration of experimental data on an exemplary diaphragm under one embodiment;

FIG. 5E is an illustration of experimental data on an exemplary diaphragm under one embodiment;

FIG. 6A is an illustration of experimental data related to an exemplary probe under one embodiment;

FIG. 6B is an illustration of experimental data related to an exemplary probe under one embodiment;

FIGS. 7A and 7B are illustrations of cassettes for use with an eye treatment system under one embodiment; and

FIG. 8. Is an illustration of a cassette receiving area for use with an eye treatment system under one embodiment.

### DETAILED DESCRIPTION OF THE INVENTION

It is to be understood that the figures and descriptions of the present invention have been simplified to illustrate elements that are relevant for a clear understanding of the present invention, while eliminating, for the purpose of clarity, many other elements found in typical surgical, and particularly optical surgical, apparatuses, systems, and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to the disclosed elements and methods known to those skilled in the art.

The figures and descriptions provided herein may have been simplified to illustrate aspects that are relevant for a clear understanding of the herein described apparatuses, systems, and methods, while eliminating, for the purpose of clarity, other aspects that may be found in typical similar devices, systems, and methods. Those of ordinary skill may thus recognize that other elements and/or operations may be desirable and/or necessary to implement the devices, systems, and methods described herein. But because such elements and operations are known in the art, and because they do not facilitate a better understanding of the present disclosure, for the sake of brevity a discussion of such elements and operations may not be provided herein. However, the present disclosure is deemed to nevertheless include all such elements, variations, and modifications to the described aspects that would be known to those of ordinary skill in the art.

Embodiments are provided throughout so that this disclosure is sufficiently thorough and fully conveys the scope of the disclosed embodiments to those who are skilled in the art. Numerous specific details are set forth, such as examples of specific components, devices, and methods, to provide a thorough understanding of embodiments of the present disclosure. Nevertheless, it will be apparent to those skilled in the art that certain specific disclosed details need not be employed, and that exemplary embodiments may be embodied in different forms. As such, the exemplary embodiments should not be construed to limit the scope of the disclosure. As referenced above, in some exemplary embodiments, well-known processes, well-known device structures, and well-known technologies may not be described in detail.

A surgical cassette, also referred to as a medical pack, a fluidic cassette, or simply, a cassette, is used to facilitate irrigation and aspiration during surgical procedures, such as phacoemulsification surgery. The surgical cassette may be inserted and mounted to a surgical console and become part of an overall phacoemulsification surgery system. The surgical cassette may perform a myriad of functions, such as effluent material collection, tube pressure sensing, and control the flow of fluid through tubing encased within the cassette and between a surgical handpiece and a surgical console.

A surgical cassette may comprise a front plate and a back plate, and may also include a gasket at least partially there between. Other configurations of the cassette are contemplated with the present invention. Molded within either/or the front plate and the back plate may be pathways for fluid flow and/or for tubing to be inserted thereby creating desired pathways for the tubing around the gasket. In an embodiment where there is a gasket, the gasket may comprise one or more valves and one or more sensors to promote fluid flow through the tubing along the desired pathways. In another embodiment, a surgical cassette may have no tubing and/or gasket. In an embodiment where there is no gasket, any valves known in the art may be used, e.g., a rotary valve.

Surgical cassettes may utilize different types of sensors to monitor pressure of certain fluid lines during the surgical process. Other single use cassettes may use a low-cost pressure diaphragm on the cassette with a console mounted Linear Variable Differential Transformer (LVDT) to measure the deflection of the pressure diaphragm with either a low rate spring pushing the LVDT against the surface of the pressure diaphragm or a magnet coupling the LVDT to the surface of the diaphragm, or a combination of both a spring and magnet. The spring force and/or friction force associated with movement of the LVDT sensing element reduces the accuracy and repeatability of this type system. Other systems may use laser triangulation displacement sensors to measure the deflection of a pressure diaphragm. In addition, other systems may use a ferromagnetic element in the cassette which couples to a magnetic element in the console, which may be coupled with a strain gauge.

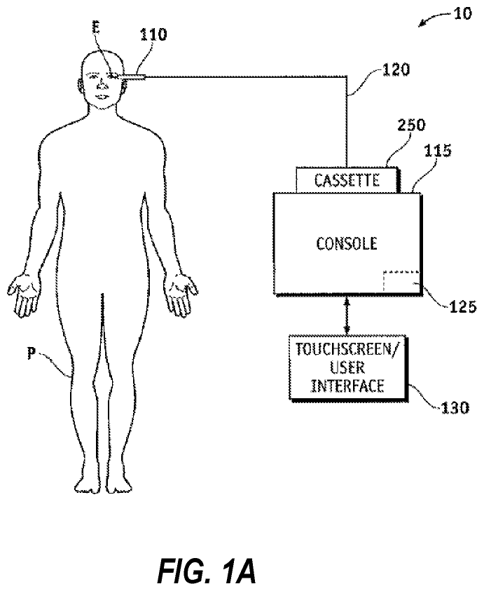

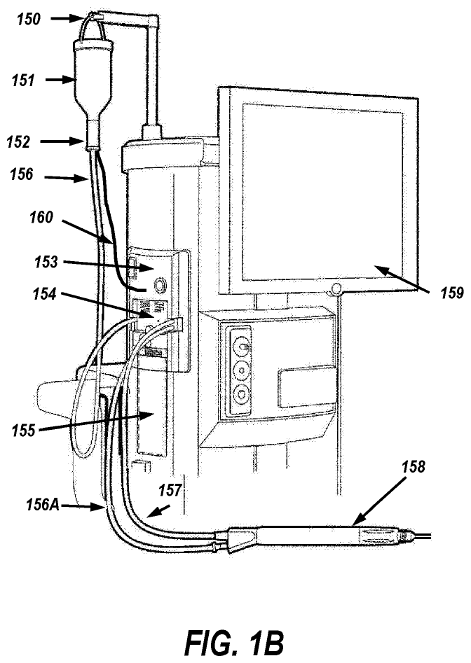

Referring now to FIG. 1A, a system 10 for treating an eye E of a patient P generally includes an eye treatment probe handpiece 110 coupled with a console 115 by a cassette 250. Handpiece 110 generally includes a handle for manually manipulating and supporting an insertable probe tip. The probe tip has a distal end which is insertable into the eye, with one or more lumens in the probe tip allowing irrigation fluid to flow from console 115 and/or cassette 250 into the eye. Aspiration fluid may also be withdrawn through a lumen of the probe tip, with console 115 and cassette 250 generally including a vacuum aspiration source, a positive displacement aspiration pump, or both to help withdraw and control a flow of surgical fluids into and out of eye E. As the surgical fluids may include biological materials that should not be transferred between patients, cassette 250 will often comprise a sterilizable (or alternatively, disposable) structure, with the surgical fluids being transmitted through flexible and/or rigid conduits 120 of cassette 250 that avoid direct contact in between those fluids and the components of console 115.

When a distal end of the probe tip of handpiece 110 is inserted into an eye E, for example, for removal of a lens of a patient P with cataracts, an electrical conductor and/or pneumatic line (not shown) may supply energy from console 115 to an ultrasound transmitter of handpiece 110, a cutter mechanism, or the like. Alternatively, handpiece 110 may be configured as an irrigation/aspiration (FA) and/or vitrectomy handpiece. Also, the ultrasonic transmitter may be replaced by other means for emulsifying a lens, such as a high energy laser beam. The ultrasound energy from handpiece 110 helps to fragment the tissue of the lens, which can then be drawn into a port of the tip by aspiration flow. So as to balance the volume of material removed by the aspiration flow, an irrigation flow through handpiece 110 (or a separate probe structure) may also be provided, with both the aspiration and irrigation flows being controlled by console 115.

To avoid cross-contamination between patients without incurring excessive expenditures for each procedure, cassette 250 and its flexible conduits 120 may be disposable. However, the flexible conduit or tubing may be disposable, with the cassette body and/or other structures of the cassette being sterilizable. Cassette 250 may be configured to interface with reusable components of console 115, including, but not limited to, peristaltic pump rollers, a Venturi or other vacuum source, a controller 125, and/or the like.

Console 115 may include controller 125, which may include an embedded microcontroller and/or many of the components common to a personal computer, such as a processor, data bus, a memory, input and/or output devices (including a user interface 130 (e.g. touch screen, graphical user interface (GUI), etc.), and the like. Controller 125 will often include both hardware and software, with the software typically comprising machine readable code or programming instructions for implementing one, some, or all of the methods described herein. The code may be embodied by a tangible media such as a memory, a magnetic recording media, an optical recording media, or the like. Controller 125 may have (or be coupled with) a recording media reader, or the code may be transmitted to controller 125 by a network connection such as an internet, an intranet, an ethernet, a wireless network, or the like. Along with programming code, controller 125 may include stored data for implementing the methods described herein, and may generate and/or store data that records parameters corresponding to the treatment of one or more patients.

Referring now to FIG. 1B, a simplified surgical console is illustrated, where a fluid path may be demonstrated under an exemplary embodiment. In this example, an irrigation source 151 may be configured as a bottle or bag hanging from an IV pole hanger 150. It is understood by those skilled in the art that, while an integrated IV pole is illustrated, other configurations, utilizing standalone/static IV poles, pressurized infusion sources, and/or other suitable configurations, are contemplated by the present disclosure.

An exemplary irrigation path for fluid may be realized via tubing cassette 154 having cassette tubing interface or receptacle 153, which receives fluid from irrigation source 151 via drip chamber 152. Irrigation line 156A and aspiration line 157 are coupled to handpiece 158. Irrigation fluid may flow from drip chamber 152 through the irrigation tubing into tubing cassette 154. Irrigation fluid may then flow from the tubing cassette through handpiece irrigation line 156A which may be coupled to an irrigation port on handpiece 158. Aspirated fluid may flow from the eye through the handpiece aspiration line 157 back to tubing cassette 154 and into a waste collection bag 155. A touch screen display 159 may be provided to display system operation conditions and parameters, and may include a user interface (e.g., touch screen, keyboard, track ball, mouse, etc. —see controller 125 of FIG. 1A) for entering data and/or instructions to the system of FIG. 1B.

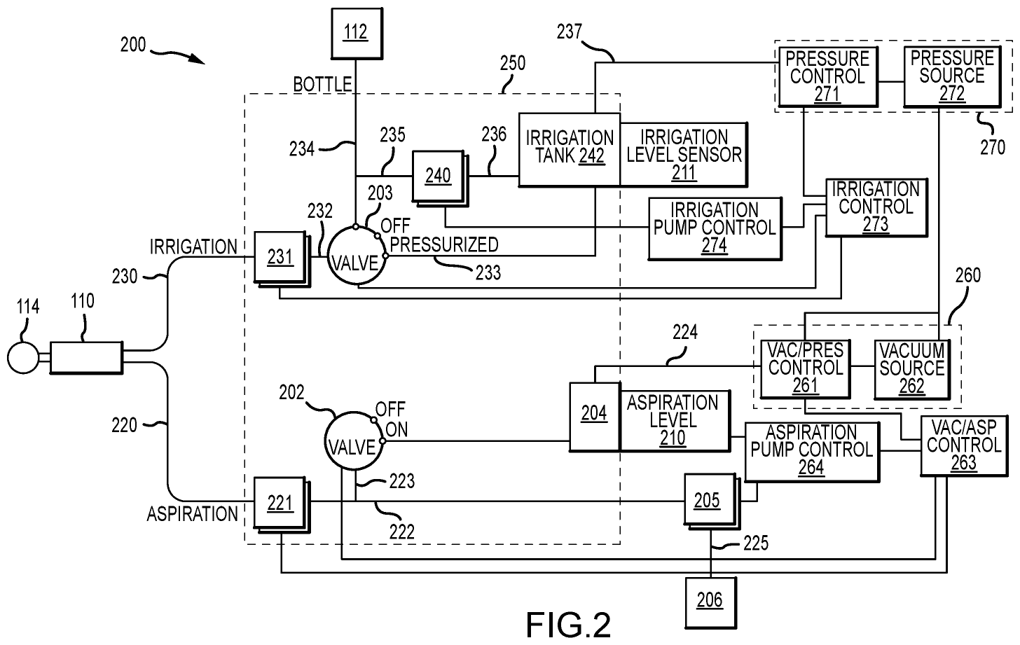

Referring to FIG. 2, an exemplary cassette system showing some of the components and interfaces that may be employed in a phaco system, such as ones illustrated in FIGS. 1A-B. Handpiece 110 may be connected to (or coupled with) the input side of sensor 221, typically by fluid pathways such as fluid pathway 220. Sensor 221 may be a pressure, flow, or a vacuum sensor that measures pressure, flow or vacuum, respectively. In a preferred embodiment, sensor 221 is a pressure sensor. The output side of sensor 221 is connected to valve 202 and also connected to pump 205 within cassette 250 via fluid pathway 222. Valve 202 may be any known valve in the art, e.g., flow selector valve, rotary valve, etc. Valve 202 may also be coupled with pump 205. The exemplary embodiment may configure valve 202 to interface between handpiece 110, vacuum tank 204, pump 205, which may be a peristaltic pump but may be another type of pump, and collection 206. In this configuration, the system may operate valve 202 to connect handpiece 110 with vacuum tank 204 based on signals received from console 115 resulting from the surgeon's input to user interface 130. In an embodiment, the handpiece 110 is always connected to pump 205 and valve 202 and may be toggled to connect or disconnect the handpiece 110 to the tank 204. As discussed herein in greater detail, an aspiration level sensor 210 may be communicatively coupled to vacuum tank 204.

The valve 202 illustrated in FIG. 2 may provide a connection between vacuum tank 204 and fluid pathway 222. The exemplary embodiment is not limited to one valve and may be realized using two valves each having at least two output ports, possibly connected together to provide the functionality described herein. For example, a pair of two valves may be configured in a daisy chain arrangement, where the output port of a first valve is directly connected to the input port of a second valve. Console 115 may operate both valves together to provide three different flow configurations. For example, using two valves, valve one and valve two, valve one may use output port one, which is the supply for valve two. Valve two may connect to one of two ports providing two separate paths. When valve one connects its input port to its second output port rather than the output port that directs flow to the second valve, a third path is provided. It is also envisioned that valve 202 may be or comprise one or more pinch valves. The one or more pinch valves may be located along fluid pathway 220, 222 and/or 223, or any other fluid pathway as discussed herein.

Console 115 may also comprise vacuum pressure center 260 which may provide a vacuum through fluid pathway 224 to vacuum tank 204. The vacuum provided through fluid pathway 224 may be regulated by control module 261 based on signals received from aspiration control module 263 which may result from the surgeon's input to user interface 130 and/or based on other signals received from sensor 221. Aspiration control module 263 may also control pump control 264 and allow for operation of pump 205 for the movement of fluid from both the handpiece 110 and the vacuum tank 204 to collector 206 via pathway 225.

In the configuration shown, vacuum pressure center 260 includes a vacuum source 262, such as a venturi pump and an optional control module 261 (and valve (not shown)), but other configurations are possible. In this arrangement, vacuum pressure center 260 may operate to remove air from the top of vacuum tank 204 and deliver the air to atmosphere (not shown). Removal of air from vacuum tank 204 in this manner may reduce the pressure within the tank, which may reduce the pressure in the attached fluid pathway 220, to a level less than the pressure within eye 114. A lower reservoir pressure connected through valve 202 may cause fluid to move from the eye, thereby providing aspiration.

Thus, while a single valve 202 is illustrated in FIG. 2 associated with aspiration, it is to be understood that this illustration represents a valve arrangement, including one or more valves (e.g. flow selector valve, rotary valve, or the like) performing the functionality described herein, and is not limited to a single device or a single valve. In the exemplary sensor 221, a strain gauge or other suitable component may communicate or signal information to console 115 to provide an amount of vacuum sensed in the handpiece fluid pathway 220. Console 115 may determine the actual amount of vacuum present based on the communicated information.

Sensor 221 monitors the pressure of fluid flowing into and out of the line and can be used to determine when fluid flow should be reversed, such as encountering a certain pressure level (e.g. in the presence of an occlusion), and based on values obtained from the sensor 221, the system may control selector valve 202 and the pumps illustrated. It is to be understood that while components presented in FIG. 2 and other drawings of the present application are not shown connected to other system components, such as console 115, they are in fact connected for the purpose of monitoring and control of the components illustrated.

With respect to sensor 221, emergency conditions such as a dramatic drop or rise in pressure may result in a type of fail-safe operation. The exemplary embodiment employs sensor 221 to monitor the flow conditions and provide signals representing flow conditions to the system such as via console 115 for the purpose of controlling components shown including but not limited to selector valve 202 and the pumps shown. The fluid pathways or flow segments of surgical cassette system 200 may include the fluid connections, for example flexible tubing, between each component represented with solid lines in FIG. 2. In an embodiment, the fluid connections may include molded fluid channels.

Handpiece 110 may be connected to (or coupled with) the output side of irrigation sensor 231, typically by fluid pathways such as fluid pathway 230. Sensor 231 may be a pressure, flow, or a vacuum sensor that measures pressure, flow or vacuum, respectively. In a preferred embodiment, sensor 231 is a pressure sensor. The input side of irrigation sensor 231 may be connected to valve 203 within cassette 250 via fluid pathway 232. Valve 203 may be any known valve in the art, e.g., flow selector valve, rotary valve, etc. The exemplary embodiment may configure valve 203 to interface between handpiece 110, irrigation tank 242, pump 240, which may be a peristaltic pump but may be another type of pump, and irrigation fluid source 112. In this configuration, the system may operate valve 203 to connect handpiece 110 with gravity feed or pressurized irrigation based on signals received from console 115 resulting from the surgeon's input to user interface 130.

The valve 203 illustrated in FIG. 2 may provide a connection between irrigation tank 242, irrigation fluid source 112, and fluid pathway 232. The exemplary embodiment is not limited to one valve and may be realized using two valves each having at least two output ports, possibly connected together to provide the functionality described herein. For example, a pair of two valves may be configured in a daisy chain arrangement, where the output port of a first valve is directly connected to the input port of a second valve. Console 115 may operate both valves together to provide three different flow configurations. For example, using two valves, valve one and valve two, valve one may use output port one, which is the supply for valve two. Valve two may connect to one of two ports providing two separate paths. When valve one connects its input port to its second output port rather than the output port that directs flow to the second valve, a third path is provided. It is also envisioned that valve 203 may be or comprise one or more pinch valves. The one or more pinch valves may be located along fluid pathway 230, 232, 233, 234 and/or 235, or any other fluid pathway as discussed herein.

Console 115 may also comprise irrigation pressure center 270 which may provide a positive pressure through fluid pathway 237 to irrigation tank 242. Irrigation pressure center may include pressure control 271 and pressure source 272. The pressure provided through fluid pathway 237 may be regulated by control module 271 based on signals received from irrigation control module 273 which may result from the surgeon's input to user interface 130 and/or based on other signals received from vacuum pressure sensor 231. Irrigation control module 273 may also control irrigation pump control 274 and allow for operation of pump 240 for the movement of fluid from irrigation fluid source 112 to collector irrigation tank 242 via pathway 236. In addition, an irrigation level sensor 211 may be communicatively coupled with the irrigation tank 242.

While a single valve 203 is illustrated in FIG. 2 associated with irrigation, it is to be understood that this illustration represents a valve arrangement, including one or more valves performing the functionality described herein, and is not limited to a single device or a single valve. In the exemplary irrigation sensor 231, a strain gauge or other suitable component may communicate or signal information to console 115 to provide an amount of pressure sensed in the handpiece fluid pathway 230. In another embodiment, depending upon the sensor used, an amount of vacuum or flow may be sensed in the handpiece fluid pathway 230 and communicated to console 115. Console 115 may determine the actual amount of pressure present based on the communicated information.

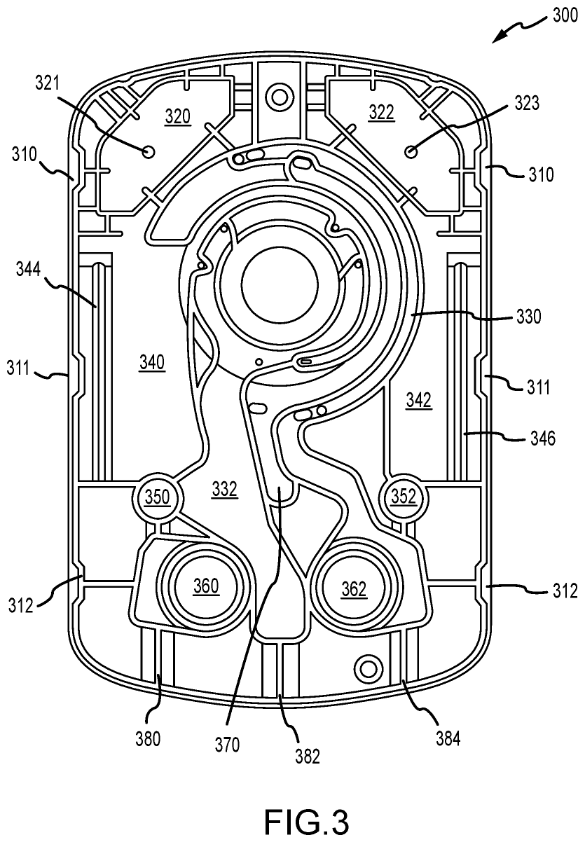

FIG. 3 illustrates an exemplary surgical cassette showing some of the features which may be employed in a phaco system. Cassette 300 may include a series of detents, also referred to as notches or catch surfaces, along its outer edge for receiving at least a portion of a retention device which may be associated with a surgical console to facilitate the retaining of the cassette to the console and to at least partially assist in properly seating the cassette in the portion of the console meant to receive the cassette. As illustrated in FIG. 3, a cassette may include at least three sets of detents capable of accepting an attachment means provide by the console, such as, for example, upper detents 310, center detents 311, and lower detents 312. As will be described in greater detail below, the detents may be operated on in tandem or in a piecemeal fashion by a retention device of the surgical console.

An exemplary cassette may also include at least one pressurized fluid inlet 321 which may be in fluid communication with at least one filter within filter cavity 320. The pressurized fluid, for example, air, may be supplied to the cassette through fluid inlet 321 and introduced into pressurized irrigation tank 340 and may be in further communication with pressure sensor 360. There may similarly be at least one vacuum inlet 323 which may be in fluid communication with at least one filter within filter cavity 323. The vacuum applied through vacuum inlet 323 may be in communication with vacuum tank 342 and may be in further communication with aspiration channel 330 and aspiration channel 370. Each of the pressurized irrigation tank 340 and vacuum tank 342 may include a level sensing device 344 and 346, respectively.

Irrigation fluid may enter the cassette through inlet 382 and may enter irrigation bladder 332. Irrigation valve 350 controls the flow of irrigation fluid and may allow for gravity fed irrigation fluid to be supplied to irrigation outlet 380 from irrigation bladder channel 332 or pressurized irrigation fluid from pressurized irrigation tank 340. In either instance, and even when irrigation valve 350 is in the “off” position relative to both irrigation fluid sources, the amount of pressure associated with the delivery of the irrigation fluid may be measured by irrigation sensor 360. Similarly, aspiration pressure may be measured by the aspiration sensor 362 in close proximity to aspiration inlet 384. Aspiration fluid which may enter though aspiration inlet 384 may enter vacuum bladder channel 330 under pressure produced by at least one peristaltic pump, for example, and may also enter vacuum tank 342 under the influence of at least a partial vacuum through valve 352.

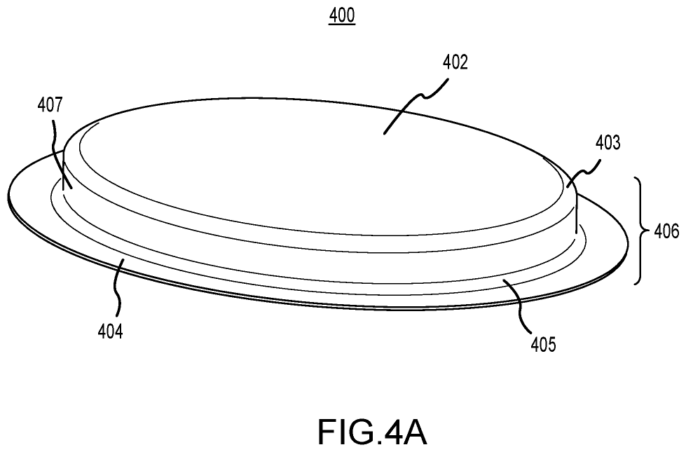

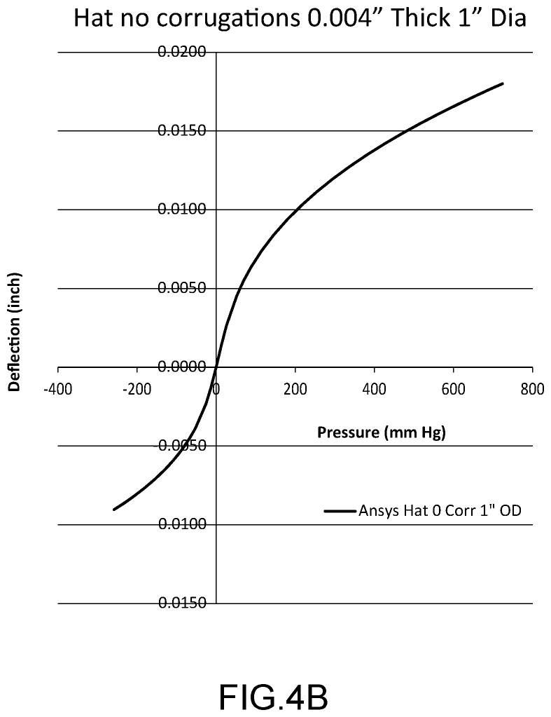

In an embodiment of the present invention, a non-contacting pressure sensor may comprise a pressure sensing diaphragm, in which the center section deflects based on the pressure acting on it, and an eddy current sensor probe, which may measure the distance between the probe face and the diaphragm. As illustrated in FIG. 4A, a diaphragm 400 may be formed having a “hat” profile 406 which may further comprise a mounting portion 404, a side portion 407, and a top portion 402. The mounting portion 404 and side portion 407 may be separated by a first radius portion 405, while side portion 407 and top portion 402 may be separated by second radius portion 403. For example, when a higher pressure acts on one side of the diaphragm 400, such as the inside portion of the “hat” profile 406, the diaphragm 400 may deflect towards an eddy current probe (not shown) which may be in close proximity to top portion 402. Similarly, when a lower pressure acts on the diaphragm, the top portion may deflect away from the current probe.

As illustrated in the two examples above, the present invention allows both internal pressures and vacuums to be measured by sensor 400. As further illustrated in FIG. 4B, the deflection of the top portion 402 of diaphragm 400 may be measured with respect to the pressure exerted on the diaphragm. As illustrated in FIG. 4A, a diaphragm may have, for example, a smooth and continuous top portion without corrugations.

In an embodiment of the present invention, a pressure sensing diaphragm may mounted in a single use cassette while an eddy current probe and associated sensing system electronics (not shown) are mounted in the surgical console. As illustrated in FIG. 3, a diaphragm may comprise a part of irrigation sensor 360 and aspiration sensor 362, for example. In an embodiment of the present invention, each of irrigation sensor 360 and aspiration sensor 362 may functionally comprise or be associated with an eddy current probe as described more fully herein. In an embodiment, the eddy current probe may be located in the console and communicatively couple with irrigation sensor 360 and/or aspiration sensor 362. When used with a surgical cassette, the diaphragms may contain no springs or other devices with associated friction wherein the use of such mechanical features may add uncertainty in the pressure measurement. Thus, in an embodiment of the present invention, there is no contact between the pressure sensing diaphragm and an eddy current probe which may be positioned to measure any movement of the diaphragm.

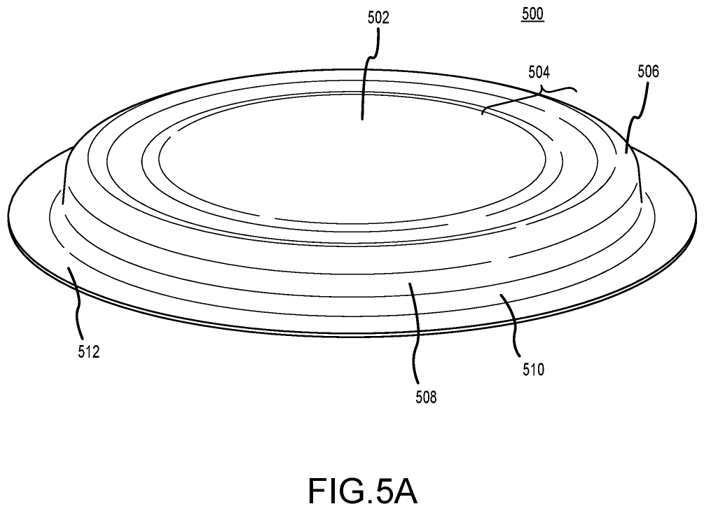

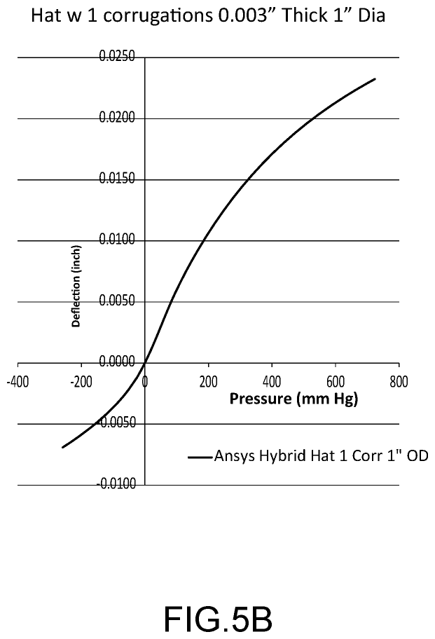

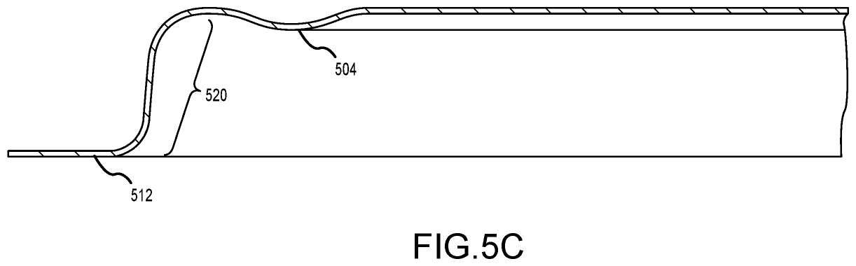

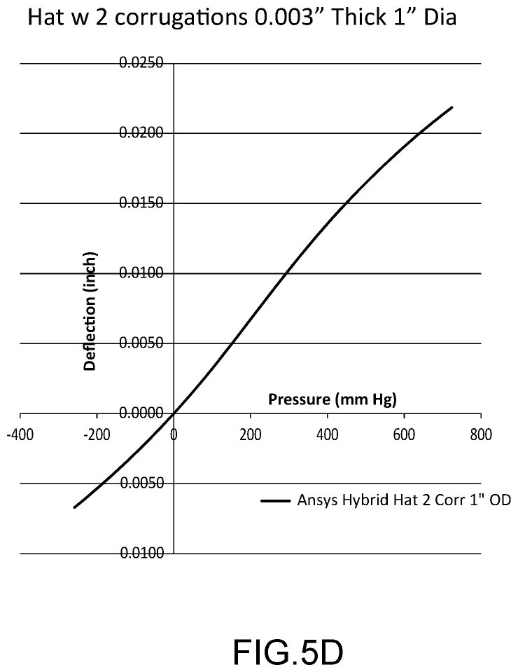

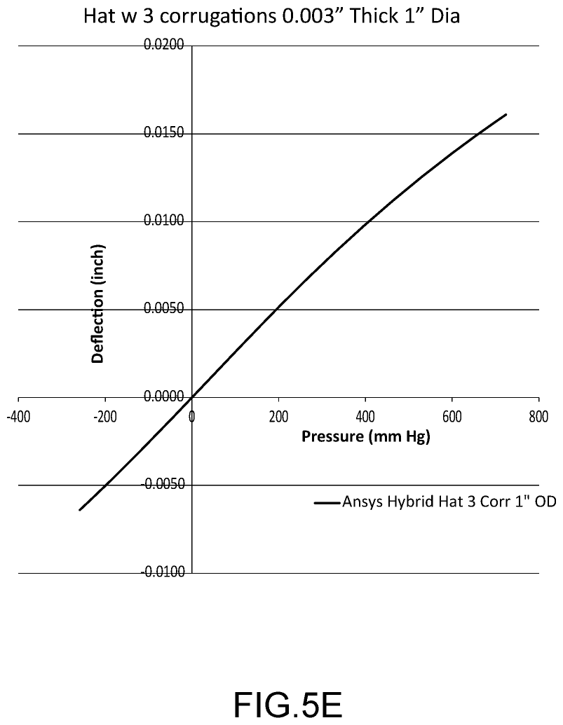

In an embodiment of the present invention, a diaphragm may be about 0.003″ thick and about 1.0″ in diameter, for example, and may be stamped from a sheet of magnetic stainless steel, such as 17-7 TH1050, which may be sensitive to pressures acting on each side of the diaphragm. Any metal known in the art that is suitable for such use is contemplated as a material for the diaphragm. In an embodiment of the present invention, a portion of the diaphragm may include corrugated aspects which may allow the diaphragm to be more responsive to a wider range of exerted pressure. As illustrated in FIG. 5A, diaphragm 500 may include a mounting portion 512, a side portion 508 and a top portion 502. A first radius portion 510 may be between mounting portion 512 and side portion 508 and second radius portion 506 may be between side portion 508 and top portion 502. Top portion 502 may further comprise corrugated portion 504 which may be located proximate to the intersection of second radius portion 506 and top portion 502. The corrugated portion 504 may include one or more nonlinear shapes relative to the plane of top portion 502 and may, for example, be less than fifty percent of the total area of top portion 502. The shaping of corrugated portion 504 may take place during the stamping process as diaphragm 500 is shaped. The corrugating may improve linearity of the pressure deflection response and improve sensitivity in the extremes of the measurement range of diaphragm 500 as is illustrated in FIG. 5B.

As illustrated in FIG. 5C, a dimensioned cross section of a portion of corrugated diaphragm may include a mounting portion 512, a profile 520, and at least one corrugated portion 504. Such a diaphragm may provide a good response over the desired pressure measurement range and may provide acceptable resolution and/or improved accuracy and resolution around zero pressure while having improved sensitivity at the extremes of the measurement range as compared to diaphragms without a corrugation portion.

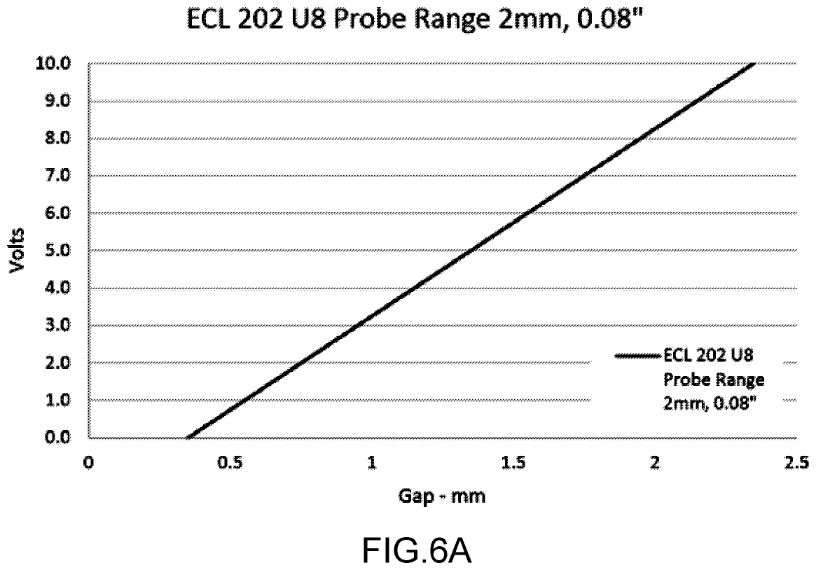

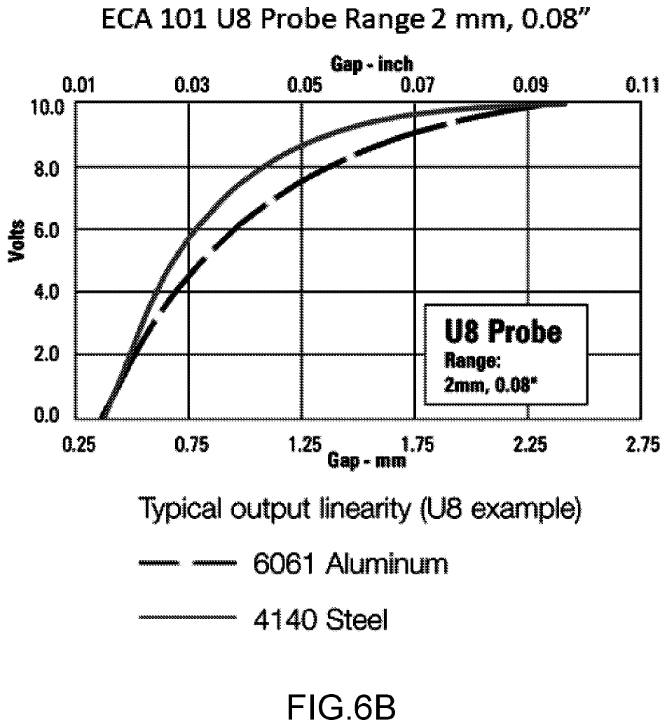

For a given displacement resolution and displacement accuracy, a pressure versus deflection response curve with a steeper slope will have improved pressure resolution and pressure accuracy as compared to a response curve with a lower slope. Furthermore, in an embodiment, an eddy current sensing system with a non-linear response curve, as shown, for example, in FIG. 6B, will tend to increase the sensitivity around zero pressure due to the steeper responses at smaller displacements and reduce the sensitivity of higher pressures due to the reduced slope at larger displacements. As shown in FIG. 4B, the response curve of a diaphragm with no corrugations has a very non-linear response curve which results in poor sensitivity at the extremes of the measurement range compared to pressures around zero. It is desirable to increase sensitivity at the pressure extremes. As illustrated in FIG. 5D, the response curve of a diaphragm with at least one corrugation provides has increased linearity and increased slope at the extremes of the pressure measurement range when compared to a diaphragm with no corrugations. Similarly, as illustrated in FIG. 5E, the response curve of a diaphragm with at least 3 corrugations provides an even more linear response curve and increased sloped at the extremes.

In an embodiment of the present invention, a sensor utilizing an alternating magnetic field may generate Eddy currents associated with a metallic interface. As would be appreciated by those skilled in the art, Eddy-Current sensors operate with magnetic fields and can create an alternating current in a sensing coil located near the end of the sensor. This creates an alternating magnetic field which induces small currents in the target material; these currents are called eddy currents. The eddy currents create an opposing magnetic field which resists the field being generated by the sensor coil. The interaction of the generated magnetic fields may be dependent on the distance between the sensor and the target material. As the distance changes, the sensor may record a change in the field interaction and produce a voltage output which is proportional to the change in distance between the sensor and target material. The target material surface, generally, for example, must be at least three times larger than the sensor diameter for normal, calibrated operation.

An eddy current sensor for use with the present invention may comprise a coil of wire with an air core and protected by a non-conductive and non-magnetic protective cover. A magnetic field may be induced in the coil by an alternating current driven by the eddy current driver component of the sensing system electronics associated with the surgical console. This induced alternating magnetic field reacts with the metal of the pressure sensitive diaphragm creating eddy currents in the diaphragm material. The eddy currents create an opposing magnetic field which resists the field being generated by the sensor coil. The interaction of the magnetic fields may be dependent on the distance between the eddy current sensor and the pressure sensing diaphragm. As this distance changes, the eddy current driver electronics may sense the change in the field interaction and produce a voltage output which is proportional to the change in distance between the probe and pressure sensing diaphragm. For good response of the eddy current sensing system, there must be adequate geometry and material properties associated with the diaphragm, such as, for example, thickness, diameter, magnetic permeability and conductance; for the eddy current sensing system to measure the distance between the sensor and diaphragm accurately.

The use of an eddy current sensor has many advantages as compared to other noncontact sensing technologies such as optical, and/or laser technologies. For example, such sensors easily tolerance dirty environments, are generally not sensitive to material in the gap between the sensor and the target material, can be less expensive and much smaller than laser interferometers, for example, and can be less expensive than capacitive sensors. In an embodiment of the present invention, a thin magnetic diaphragm may be used for precise and accurate pressure measurements by the eddy current displacement sensor system used in the present invention. In embodiments of the present invention, other metallic materials could be used including nonmagnetic materials such as aluminum or titanium. In some cases, with non-magnetic materials, the diaphragm may have to be relatively thick for the eddy current sensor to be able to adequately sense the diaphragm. Thicker diaphragms may be mechanically insensitive to the desired pressure range to be sensed. In these cases, a separate material target could be mounted on the center of the diaphragm so that it allows for a thinner more pressure sensitive diaphragm and allowing the eddy current sensor to measure the distance from the affixed target. As illustrated in Table 1 below, various thicknesses for nonmagnetic materials that are known in the art may be used with the present invention:

TABLE 1

| | | | | Minimum | |
|---|---|---|---|---|---|
| | | | | Thickness | |
| Material | P | pr | Probe (1 MHz) | mm | mils |
| Silver | 1.59 | 1 | U3 to U8 (1.0) | 0.19 | 7.5 |
| | | | U12 to U50 (.05) | 0.27 | 10.6 |
| Copper | 1.71 | 1 | U3 to U8 (1.0) | 0.2 | 7.8 |
| | | | U12 to U50 (.05) | 0.28 | 11 |
| Gold | 2.21 | 1 | U3 to U8 (1.0) | 0.22 | 8.8 |
| | | | U12 to U50 (0.5) | 0.32 | 12.5 |
| Aluminum | 2.65 | 1 | U3 to U8 (1.0) | 0.25 | 9.7 |
| | | | U3 to U8 (10.0) | 0.35 | 13.7 |
| Zinc | 5.97 | 1 | U3 to U8 (1.0) | 0.37 | 14.5 |
| | | | U12 to U50 (0.5) | 0.52 | 20.5 |
| 304 SST | 72 | 1.01 | U3 to U8 (1.0) | 1.27 | 50.2 |
| | | | U12 to U50 (0.5) | 1.8 | 70.9 |
| Lead | 20.8 | 1 | U3 to U50 (0.5) | 0.69 | 27.1 |
| | | | U12 to U50 (0.5) | 0.97 | 38.3 |
| Brass | 6.4 | 1 | U3 to U8 (1.0) | 0.38 | 15 |
| | | | U12 to U50 (0.5) | 0.54 | 21.3 |
| Tin | 11.5 | 1 | U3 to U8 (1.0) | 0.51 | 20.1 |
| | | | U12 to U50 (0.5) | 0.72 | 28.5 |
| Titanium | 47 | 1 | U3 to U8 (1.0) | 1.03 | 40.7 |
| | | | U12 to U50 (0.5) | 1.46 | 57.6 |

As illustrated in FIGS. 6A and 6B, response curves for two different eddy current sensor systems from Precision Lion show they may be both linear and non-linear. In FIG. 6A, a Lion Precision ECL 202 eddy current displacement sensor system is used and has a 70 nm resolution for magnetic targets and 40 nm resolution for nonmagnetic targets. The output response has been linearized over the complete range with a tolerance of 0.2% nonlinearity and the unit is typically calibrated for the sensing target material and geometry. In FIG. 6B, a more economical eddy current displacement sensor system, a Lion Precision ECA 101, which has less complex electrical signal conditioning that has a nonlinear output response is illustrated. Either Eddy current displacement systems may be used with the present invention. More specifically, having a nonlinear response curve for the eddy current sensor system does not necessarily reduce the pressure sensor accuracy much beyond the effects of slightly reduced sensitivity due to the response curves reduced slope at larger displacements. This is due in part to a cassette suitable for use with the present invention may have a tolerance of approximately ±0.005 inch location accuracy in the surgical console and, through sensor calibration, may achieve a desired pressure accuracy (which may translate to a displacement accuracy of ±<1 micron) between the cassette and console. In typical usage, a single use cassette may be first mounted into the console and once rigidly affixed to the console, the cassette pressure diaphragms may be then calibrated over a desired operating range.

In an embodiment of the present invention, eddy currents may react against an induced magnetic field generated by a coil of the probe and change the complex impedance of the magnetic circuit. For adequate measurements to be made by the system, the diaphragm must be large enough in terms of thickness and diameter. Table 1, illustrated above, presents the minimum thickness of a magnetic “target” to be measured. From this table, relatively thin sections of magnetic materials may yield a good measurement In addition to magnetic permeability, a material's electrical resistivity and the eddy current sensor driver oscillation frequency are important. To calculate the minimum thickness required for a given eddy current measurement system and material the following equations can be used to determine the materials skin depth and approximate the required thickness based on three times the skin depth:

Calculating Minimum Thickness:

Minimum target thickness is three times the target material's “skin-depth.”

Skin-depth (δ):

δ=1.98[ρ/(fμr)]\hat{ }½ inches

δ=50.3 [ρ/(fμr)]\hat{ }½ mm

minimum target thickness=3δ

where:

ρ=electrical resistivity, μ-ohm-cm

f=oscillation frequency, hertz

μr=magnetic permeability

Field density decreases exponentially with depth (1/e). At three skin-depths eddy current density is about 5% of the surface density. Three skin-depths is the minimum target thickness suitable for optimum performance.

In a first embodiment of the present invention, a diaphragm may be made of 17-7 stainless steel (17-7 SS) which has undergone at least one heat treatment process after being formed by stamping or other suitable manufacturing process. As would be appreciated by those skilled in the art, 17-7 stainless steel is classified as a Precipitation Hardening Stainless Steel and is typically used for applications requiring high material strength and a moderate level of corrosion resistance. 17-7 SS is typically supplied from the steel mill in the annealed condition (Condition A) where the steel is in an austenitic phase of steel. In this annealed austenitic condition, the steel is in a Face-Centered Cubic (FCC) form and is non-magnetic and cannot be accurately measured by the eddy current sensing system of the present invention. After forming, a diaphragm may undergo a low temperature heat treat such as RH 950 or TH 1050 which converts some of the austenite (FCC) into martensite which is a Body-Centered Tetragonal (BCT) form, similar to the ferrite (BCC) phase of steel. This conversion of approximately 50% to 90% of the austenite into martensite, strengthens the diaphragm material allowing for a greater range of pressure measurement and also makes the material magnetic so that its deflection can easily be measured by the eddy current distance sensor of the present invention.

In an embodiment of the present invention, a flat diaphragm, one with no visually determinable mounting portion in a similar fashion as with a “hat” diaphragm, may be constructed of 17-7 SS and may be about 0.002 inches thick. A flat diaphragm may have a very nonlinear response to the differential pressure acting on it and may be sensitive to pressures around zero differential pressure. As the pressure differential magnitude increases, the pressure response is reduced with less deflection as the pressure changes. In testing with a flat diaphragm, it was also found that the pressure response would show hysteresis. This hysteresis was shown as having slightly different pressure response going from low pressure to high pressure compared to the pressure response from high pressure to low pressure. The hysteresis may be caused by friction in the mounting on the outside annulus of the disc. As the diaphragm flexes from pressure application, a portion of the mounting portion may be pulled in towards the center of the diaphragm. Generally, a “hat” shaped diaphragm does not show the same hysteric response as shown by a flat diaphragm.

In an embodiment of the present invention, the diaphragm may be formed into a “hat” shape with one corrugation on the top face. In an embodiment of the present invention, a “hat” diaphragm with no corrugations but with a mounting flange may be used. In an embodiment of the present invention, as compared to a flat diaphragm, the active center part of a diaphragm, above the vertical section of the hat, may act as a smaller diaphragm with the vertical section of the hat allowing slight movements on the other edge of the active diaphragm, allowing the diaphragm to be slightly easier to stretch at high deflections. Such a “hat” formed diaphragm may demonstrate increased stiffness around zero pressure, which may be due to increased thickness of this diaphragm (0.004 inches vs. 0.002 inches for a flat diaphragm), while showing reduced stiffness at larger deflections.

In an embodiment of the present invention, a hat diaphragm under about 750 mm Hg differential pressure may exhibit a maximum stress around the outside of the active diaphragm where it joins the vertical hat section, which may be about 74 ksi (kilopound per square inch). This force is about 50% of the yield stress for heat treated 17-7 SS, allowing satisfactory life for the single use cassette. In an embodiment of the present invention, a “hat” shaped diaphragm with one corrugation under about 750 mm Hg differential pressure may exhibit a near maximum stress at the junction between the active diaphragm and the vertical hat section. Although such a diaphragm may be thinner than the non-corrugated hat diaphragm, it has lower maximum stress of about 70 ksi (compared to 74 ksi) and may thus have slightly longer operable life. In an embodiment of the present invention, a “hat”-shaped diaphragm with two corrugations may be stiffer around zero pressure and softer at higher pressure with a pressure response closer to a linear response over the desired measurable pressure range. The hat diaphragm with two corrugations under 750 mm Hg differential pressure may exhibit a maximum stress at the junction between the active diaphragm and the vertical hat section. Although such a diaphragm may be about 0.003″ thick, it may exhibit the lowest maximum stress of about 65 ksi (compared to 74 ksi or 70 ksi) and would thus demonstrate the longest useful operational life.

In an embodiment of the present invention, a “hat”-shaped diaphragm with three corrugations may be stiffer around zero pressure and softer at higher pressure as compared to diaphragms with fewer corrugations, with pressure measurements moving more closely to a linear response over the desired pressure range. In an embodiment of the present invention, the inner corrugation may be in the same area required for the eddy current sensor magnetic flux path. Corrugations positioned within the primary magnetic flux area may reduce the accuracy of the eddy current sensor reducing the performance of the pressure sensing system. A hat diaphragm with three corrugations under about 750 mm Hg differential pressure may exhibit a maximum stress at the junction between the active diaphragm and the vertical hat section. Although the diaphragm may be 0.003″ thick, it may have the lowest maximum stress of about 62.5 ksi (compared to 74 ksi, 70 ksi & 65 ksi), thus exhibiting the longest operational life and an ability to operate over a wide pressure range without damaging the diaphragm. As demonstrated about, the “hat” feature may serve to both improve the responsiveness of the diaphragm as compared to the flat disc, may separate the mounting function from the pressure measurement function, and may reduce the hysteresis of the diaphragm.

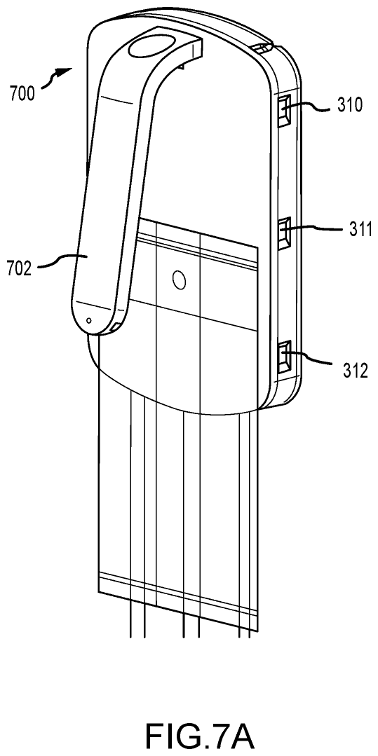

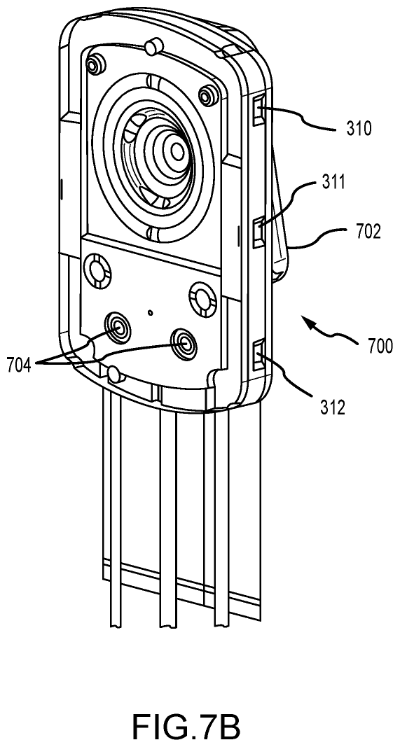

FIGS. 7A and 7B illustrate prospective views of a single use cassette 700 suitable for use with the present invention. FIG. 7A illustrates the back of single use cassette 700 which may comprise handle 702 which may help a user hold and maneuver single use cassette 700. FIG. 7B illustrates the pressure sensing diaphragms 704 integrated into a single use cassette 700. As also illustrated in FIGS. 7A and 7B, cassette 250 may generally include a cassette body 700 with at least three sets of detents capable of accepting an attachment means provided by the console, such as, for example, upper detent 310, center detent 311, and lower detent 312 and a handle portion 702. Cassette detents/notches partially define the positioning of the retention device that receives and positions cassette located within the console. In an embodiment, the cassette has two pressure sensing diaphragms 704, one for the irrigation side which supplies fluid to the surgical site, and one for the aspiration side for removing fluid from the surgical site. The irrigation pressure is typically above atmospheric pressure since the fluid requires a slight pressure to flow into the surgical site. The aspiration pressure is typically below atmospheric pressure which is required to pull the fluid away from the surgical site. Although the two pressure sensors typically operate under different conditions, both pressure sensors are calibrated over the same range from the maximum pressure which could be used on the irrigation side to the lowest pressure which could be used over the aspiration side. The eddy current displacement sensor system requires a large enough sensing range to account for the motion of the pressure sensitive diaphragms, the tolerance in motion of different diaphragms in single use cassettes, the tolerance in the mounting of the diaphragms in the cassettes as well as tolerances in the mounting of the cassettes in the console system. For our application, an eddy current sensing system with a range of 2.0 mm is used to account for all of these tolerances for a nominal 0.8 mm pressure diaphragm deflection over the desired range pressure measurement.

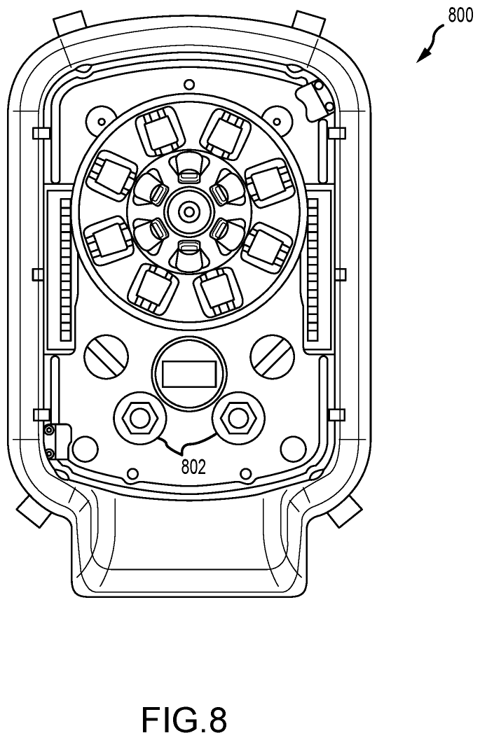

FIG. 8 shows an illustration of the console interface or cassette receptacle for mating a single use cassette with the console. The pressure sensing diaphragms on the single use cassette will center on the eddy current displacement sensor probes 802 once the cassette has been mounted to the console interface 800. As discussed above, the mounting tolerances between the cassette and console may be ±0.005 inches while the pressure sensing may require about 1 micron resolution for the desired pressure resolution. This may be accounted for by calibrating the pressure sensors on the cassette to a reference pressure sensor on the fluidic circuits of the console once the cassette is rigidly fixed to the console mounting face. In an embodiment of the present invention, the combination of a single convoluted pressure sensing diaphragm and precision Eddy current sensor may yield excellent pressure sensing capability across a desired range for both the irrigation and aspiration fluids running through a cassette.

Those of skill in the art will appreciate that the herein described apparatuses, engines, devices, systems and methods are susceptible to various modifications and alternative constructions. There is no intention to limit the scope of the invention to the specific constructions described herein. Rather, the herein described systems and methods are intended to cover all modifications, alternative constructions, and equivalents falling within the scope and spirit of the disclosure, any appended claims and any equivalents thereto.

In the foregoing detailed description, it may be that various features are grouped together in individual embodiments for the purpose of brevity in the disclosure. This method of disclosure is not to be interpreted as reflecting an intention that any subsequently claimed embodiments require more features than are expressly recited.

Further, the descriptions of the disclosure are provided to enable any person skilled in the art to make or use the disclosed embodiments. Various modifications to the disclosure will be readily apparent to those skilled in the art, and the generic principles defined herein may be applied to other variations without departing from the spirit or scope of the disclosure. Thus, the disclosure is not intended to be limited to the examples and designs described herein, but rather is to be accorded the widest scope consistent with the principles and novel features disclosed herein.

What is claimed is:

1. A pressure measurement system for use with a surgical system, comprising:
a pressure sensitive disc in communication with at least one applied pressure;
a magnetic field generator for generating at least one first magnetic field; and
at least one sensor for measuring at least one attribute of the magnetic field generator;
wherein the at least one first magnetic field at least partially creates at least one second magnetic field; and
wherein the at least one sensor produces a signal indicative of a distance between the at least one sensor and the pressure sensitive disc.

2. The system of claim 1, wherein the at least one attribute comprises voltage.

3. The system of claim 1, wherein the pressure sensitive disc is made of a non-ferrous material.

4. The system of claim 1, wherein the pressure sensitive disc comprises at least one metal selected form the group consisting of, steel, stainless steel, and aluminum.

5. The system of claim 1, wherein the pressure sensitive disc comprises at least one corrugation.

6. The system of claim 1, wherein the pressure sensitive disc comprises at least two planar portions.

7. The system of claim 1, wherein the at least one second magnetic field is indicative of an eddy current.

8. The system of claim 1, wherein the pressure sensitive disc comprises at least two corrugations.

9. The system of claim 1, wherein the pressure sensitive disc is at least 0.002 inches thick.

10. The system of claim 1, wherein the at least one sensor measures a magnetic field at a range of less than about 2.0 mm.

11. The system of claim 1, wherein at least a portion of the pressure sensitive disc positively deflects less than about 0.8 mm.

12. The system of claim 1, wherein at least a portion of the pressure sensitive disc negatively deflects less than about 0.8 mm.

13. A method for determining a pressure, comprising:
providing a first magnetic field to a metallic body to induce an at least one second magnetic field;
sensing the at least one second magnetic field using at least one sensor; and
determining the distance between the metallic body and the at last one sensor based on the at least one second magnetic field.

14. The method of claim 13, wherein the at least one second magnetic field is substantially contained in the metallic body.

15. The method of claim 13, wherein the metallic body is deformable under pressure.

16. The method of claim 13, wherein the metallic body comprises at least one metal selected form the group consisting of, steel, stainless steel, and aluminum.

17. The method of claim 13, wherein the metallic body comprises at least one corrugation.

18. The method of claim 13, wherein the metallic body comprises at least two planar portions.

19. The method of claim 13, wherein the at least one second magnetic field is indicative of an eddy current.

20. The method of claim 13, wherein the metallic body is at least 0.002 inches thick.

21. The method of claim 13, wherein the at least one sensor measures a magnetic field at a range of less than about 2.0 mm.

22. The method of claim 13, wherein at least a portion of the metallic body positively deflects less than about 0.8 mm in response to at least one pressure greater than atmosphere.

23. The method of claim 13, wherein at least a portion of the metallic body negatively deflects less than about 0.8 mm in response to at least one pressure less than atmosphere.
